(19) United States
(12) Patent Application Publication
(10) Pub. No.: US 2012/0196337 A1
(43) Pub. Date: Aug. 2, 2012

(54) HETEROGENOUS ENZYMATIC CATALYST, PREPARATION METHOD, AND USE

(76) Inventors: Nicolas Brun, Bordeaux (FR); Annick Babeau-Garcia, Pessac (FR); Clément Sanchez, Bures sur Yvette (FR); Rénal Backov, Bordeaux-Cauderan (FR)

(21) Appl. No.: 13/380,929

(22) PCT Filed: Jul. 5, 2010
(86) PCT No.: PCT/FR2010/051413

§ 371 Date: Apr. 18, 2012

(30) Foreign Application Priority Data
Jul. 6, 2009 (FR) 0954634

(51) Int. Cl. C12P 7/64; C12N 11/02; C12N 11/14

(52) U.S. Cl. 435/134; 435/176; 435/177

(57) ABSTRACT

The invention relates to a heterogenous enzymatic catalyst that takes the form of a cellular monolith consisting of a silica or organically modified silica matrix, said monolith including macropores, mesopores, and micropores, said pores being interconnected, and wherein the inner surface of the macropores is functionalized by a coupling agent selected from among silanes, said inner surface moreover having an unpurified enzyme attached thereon by means of a covalent or electrostatic bond. The invention also relates to the method for preparing said catalyst, said method comprising: a first step for preparing a solid silica impression that takes the form of a cellular monolith such as defined above; a second step for functionalizing the inner surface of the macropores via a coupling agent, selected from among silanes, by vacuum-soaking the cellular monolith by dissolving the coupling agent in an organic solvent; and a third step for vacuum-soaking the thus-functionalized monolith by means of an aqueous solution or aqueous dispersion of at least one unpurified enzyme. Finally, the invention relates to the use of such catalyst to carry out catalyzed chemical reactions.

The present invention relates to a heterogeneous enzymatic catalyst, to a process for preparing such an enzymatic catalyst, and to the use thereof for carrying out chemical reactions by enzymatic catalysis in the heterogeneous phase.

Enzymatic catalysis lies within the context of “green” chemistry and sustainable development. It enables various chemical reactions to be carried out while minimizing the use of solvents and favoring the use of bio-precursors.

Considering the high price of enzymes, especially due to their production and purification costs, it is important to be able to recover them easily from the reaction medium when the reaction that they have catalyzed is complete.

One of the solutions proposed for solving this problem consists in immobilizing the enzymes on a solid support. It is then referred to as heterogeneous enzymatic catalysis.

Heterogeneous catalysis (or contact catalysis) aims to convert liquid or gaseous reactants by using a solid catalyst. The chemical process takes place at the solid-fluid interface, owing to adsorption of the reactants on the surface of the solid.

The various enzymatic catalytic systems currently proposed predominantly consist of a solid, generally porous, support (polymer matrices, colloidal silica, calcium silicate, zeolites, zirconium, kaolinite, porous glass, alumina, etc.), on which a purified enzyme is immobilized. These systems enable the easy recovery of the enzyme when the reaction is complete. However, they have a major drawback in so far as the enzymes must first be purified before being immobilized on the solid support, which inevitably increases the cost price of these catalytic systems.

Certain authors have also proposed enzymatic heterogeneous catalysts incorporating unpurified enzymes. U.S. Pat. No. 6,004,786 describes in particular a heterogeneous enzymatic catalyst consisting of a solid support such as porous glass, bentonite, silica gels, colloidal silica, silica-alumina hydroxyapatite, calcium-phosphate gels, etc. . . . on/in which unpurified lipases are immobilized by means of a silane coupling agent having a carboxylic ester function. However, it is demonstrated in the examples from this U.S. Pat. No. 6,004,786 that the immobilization of unpurified lipases on the supports used only gives satisfactory results in terms of catalytic activity when a coupling agent having a carboxylic ester function is used. In this respect, comparative lipase immobilization tests, carried out with a coupling agent that does not have a carboxylic function, namely γ-glycidoxypropyltrimethoxysilane, also known under the trade name GLYMO, do not lead to good results in terms of catalytic activity.

Therefore, it is not possible to use any coupling agent to immobilize unpurified enzymes on/in a solid support.

It has also already been proposed, especially in U.S. Pat. No. 6,025,171, to immobilize unpurified amphiphilic enzymes, such as lipases, on sugars (starch, dextran, cellulose derivatives, etc.). However, although such supports have improved thermal stability, their repeated use involves a systematic regeneration step after each catalysis reaction, which is not practical from an industrial viewpoint and prohibits their use in a continuous enzymatic catalysis process. Furthermore, the partial or complete solubility of these supports in a polar medium (water and other polar solvents) prohibits their use in any cyclical or continuous-flow catalysis process. Lastly, their use for the production of biodiesel cannot be envisaged either in so far as the newly formed compounds are all capable of dissolving the aforementioned supports.

There is therefore a need for a heterogeneous enzymatic catalyst:
that enables the use of unpurified enzymes with a very high catalytic efficiency,
that can be reused a very large number of times without significant loss of catalytic activity,
that can be used in a polar medium and/or in a continuous catalysis process (that does not require an intermediate regeneration step), and
that can be prepared according to a process that is simple to implement, especially with conventional coupling agents.

This objective is achieved by the heterogeneous enzymatic catalyst that is the subject of the present invention and that will be described below.

One subject of the present invention is a heterogeneous enzymatic catalyst, characterized in that it is in the form of a cellular monolith consisting of a silica or organically modified silica matrix, said monolith comprising macropores having a mean size d_{A} of 1 μm to 100 μm, mesopores having a mean size d_{E} of 2 to 50 nm and micropores having a mean size d₁ of 0.7 to 1.5 nm, said pores being interconnected, and in which the internal surface of the macropores is functionalized by a coupling agent chosen from silanes to which an unpurified enzyme is attached, by means of a covalent or electrostatic bond.

Within the context of the present invention, the expression “unpurified enzyme” is understood to mean any protein material comprising at least one non-isolated enzyme that has not undergone a purification step.

The term “monolith” is understood to mean a solid object having a mean size of at least 1 mm.

As is demonstrated in the examples illustrating the present application, the use of such a monolith makes it possible to use unpurified enzymes, which is very advantageous from an economic viewpoint. Indeed, the immobilization of an unpurified enzyme, by means of a coupling agent chosen from silanes, within the macropores of such a monolith results in a heterogeneous enzymatic catalyst that has a very high catalytic activity, usually reaching 100% of the theoretical catalytic activity of the enzyme when it is used in the purified or non-immobilized state, and also a very high cyclability. The inventors have also demonstrated that when an unpurified enzyme is immobilized in such a monolith, its reaction kinetics are increased.

In this monolith, the macropore walls generally have a thickness of 0.5 to 40 μm and preferably 2 to 25 μm.

According to the invention, the micropores are present within the thickness of the macropore walls, thus rendering them microporous.

The specific surface area of the monolith is generally from 200 to 1000 m²/g approximately, preferably 300 to 700 m²/g approximately.

When the cellular monolith consists of an organically modified silica matrix, the silica bears organic groups R corresponding to the following formula (I):

—(CH₂)ₙ—R¹ (I)

in which:
−0≦n≦5, and
R¹ represents a thiol group, a C₄H₃N— pyrrole group bonded via the nitrogen to the —(CH₁)ₙ— group, an amino group that optionally bears one or more optionally substituted alkyl, alkylamino or aryl substituents, an alkyl group (preferably having 1 to 5 carbon atoms), a C₂-C₂₁ monohydroxyalkyl or polyhydroxyalkyl group, a phenyl group or a phenyl group substituted by an alkyl radical, preferably a methyl group.

In particular, the organic group R may be:
a 3-mercaptopropyl group;
a 3-aminopropyl group;
an N-(3-propyl)pyrrole group;
an N-(2-aminoethyl)-3-aminopropyl group;
a 3-(2,4-dinitrophenylamino)propyl group;
a phenyl or benzyl group;
a methyl group; or
a group of formula —(CH₂OH—CH₂OH)_{q}—CH₂OH or —(CH₂OH—CH₂OH)_{q}—CH₂CH₃ in which q is an integer ranging from 1 to 10.

The silica matrix of the cellular monolith may also comprise one or more metal oxides MO₂ in which M is a metal chosen from Zr, Ti, Th, Nb, Ta, V, W and Al. In this case, the silica matrix is a mixed matrix of SiO₂-MO₂ type. Among such mixed matrices, the matrices of SiO₂—ZrO₂ type are preferred.

When the silica matrix is a mixed matrix, the content of metal oxide MO₂ preferably represents from 10 to 50% by weight approximately relative to the weight of the silica or of the organically modified silica.

According to the invention, the bond ensuring the attachment of the coupling agent to the silica, or the R group of the silica in the case of an organically modified silica, is an iono-covalent bond.

According to one preferred embodiment of the invention, the coupling agent is chosen from the silanes chosen from the group consisting of 7-glycidoxypropyltrimethoxysilane; silyl-containing ionic liquids such as for example 1-methyl-3-(3-triethoxysilylpropyl)imidazolium chloride or 1-methyl-3-(3-triethoxysilylpropyl)imidazolium hexafluorophosphate; the silanes of formula Si(OR²)₃R³ in which R² represents a C₁-C₂ alkyl group, and R³ represents a —(CH₂OH—CH₂OH)_{q}—CH₂OH or —(CH₂OH—CH₂OH)_{q}—CH₂CH₃ group in which q is an integer ranging from 1 to 10.

Among such silanes, γ-glycidoxypropyltrimethoxysilane, also known under the name Glymo, is particularly preferred.

The nature of the enzyme that can be immobilized on the silica monolith by means of the coupling agent is not critical, as long as it comprises at least one functional group capable of reacting with a complementary functional group borne by the coupling agent in order to form an iono-covalent bond. When the coupling agent used is a silyl-containing ionic liquid, these are electrostatic bonds.

According to one preferred embodiment of the invention, the unpurified enzyme is chosen from:

i) hydrolases (class EC 3 of the classification established by the Enzyme Commission, Brussels), such as esterases (EC 3.1), and in particular carboxylic ester hydrolases (EC 3.1.1) such as lipases (EC 3.1.1.3 or triacylglycerol acylhydrolases); aminoacylases (EC 3.5.1.14), amidases (EC 3.5.1.4; EC 3.5.1.3 or ω-amidase; EC 3.5.1.11 or penicillin amidase); nitrilases (class EC 3.5.5.1) which catalyze the hydrolysis of nitriles to carboxylic acids;

ii) lyases (class EC 4) especially including carboxy-lyases (EC 4.1.1), aldehyde-lyases (EC 4.1.2) such as oxynitrilases (classes EC 4.1.2.10 and EC 4.1.2.37) catalyzing the synthesis of chiral cyanohydrins; and hydro-lyases (EC 4.2.1);

iii) isomerases (EC 5) especially including epimerases and racemases (EC 5.1.), and in particular epimerases and racemases of class EC 5.1.1 that catalyze the formation of enantiomers of amino acids; and

iv) oxidoreductases (EC. 1) especially including glucose oxidases (EC 1.1.3.4) such as Aspergillus niger glucose oxidase and peroxidases (EC 1.11.1) such as horseradish peroxidase.

According to one preferred embodiment of the invention, the unpurified enzyme is chosen from lipases of microbial or plant origin, and in particular, from Candida rugosa, Candida antarctica, Aspergillus niger, Aspergillus oryzae, Thermomyces lanuginosus, Chromobacterium viscosum, Rhizomucor miehei, Pseudomonas fluorescens, Pseudomonas cepacia, Penicillium roqueforti, Penicillium expansum and Rhizopus arrhizus lipases and wheat germ lipases.

The amount of enzymes immobilized within the catalyst in accordance with the invention may be determined by thermogravimetric analysis and by elemental analysis. According to one preferred embodiment of the invention, the amount of unpurified enzyme immobilized ranges from 3 to 40% by weight approximately and more preferably from 10 to 20% by weight approximately relative to the total weight of the catalyst.

Another subject of the present invention is a process for preparing a heterogeneous enzymatic catalyst in accordance with the invention and as defined above, said process comprising a first step of preparing a solid silica template in the form of a cellular monolith consisting of a silica or organically modified silica matrix, said monolith comprising macropores having a mean size d_{A} of 1 μm to 100 μm, mesopores having a mean size d_{E} of 2 to 50 nm and micropores having a mean size d₁ of 0.7 to 1.5 nm, said pores being interconnected, said process being characterized in that it also comprises the following steps:
a second step of functionalizing the internal surface of the macropores with a coupling agent chosen from silanes, by vacuum-impregnating the cellular monolith with a solution of the coupling agent in an organic solvent;
a third step of vacuum-impregnating the thus functionalized monolith with an aqueous solution or an aqueous dispersion of at least one unpurified enzyme.

In one preferred embodiment of the invention, the preparation of the silica template, during the first step, is carried out according to the processes as described in patent applications FR-A 1-2 852 947 and FR-A 1-2 912 400.

These processes generally consist in:
preparing an emulsion by introducing an oily phase into an aqueous surfactant solution;
adding an aqueous solution of at least one silica oxide precursor and/or at least one organically modified silica oxide precursor to the surfactant solution, before or after preparation of the emulsion;
leaving the reaction mixture to rest until said precursor has condensed; and then
drying the mixture in order to obtain the expected solid silica template.

In this case, the silica oxide or organically modified silica oxide precursor(s) may be chosen from silica alkoxides of the following formula (II):

R⁴ₚ(OR⁵)₄₋ₚSi (II)

in which:
R⁴ represents an alkyl radical having 1 to 5 carbon atoms or an aryl radical that optionally bears one or more functional groups;
R⁵ represents an alkyl radical having 1 to 5 carbon atoms or a group of the following formula (I):

—(CH₂)ₙ—R¹ (I)
in which 0≦n≦5, and R¹ is chosen from a thiol group, a C₄H₃N— pyrrole group bonded via the nitrogen to the —(CH₂)ₙ— group, an amino group that optionally bears one or more optionally substituted alkyl, alkylamino or aryl substituents, an alkyl group (preferably having 1 to 5 carbon atoms), a C₂-C₂₁ monohydroxyalkyl or polyhydroxyalkyl group, a phenyl group or a phenyl group substituted by an alkyl radical, preferably a methyl group; and
p is an integer equal to 0, 1, 2 or 3.

In one embodiment, the precursor of formula (II) comprises a single type of group of formula (I). In another embodiment, the precursor of formula (II) comprises at least two different types of groups of formula (I).

In particular, the organic group of formula (I) may be:
a 3-mercaptopropyl group;
a 3-aminopropyl group;
an N-(3-propyl)pyrrole group;
an N-(2-aminoethyl)-3-aminopropyl group;
a 3-(2,4-dinitrophenylamino)propyl group;
a phenyl or benzyl group;
a methyl group; or
a group of formula —(CH₂OH—CH₂OH)_{q}—CH₂OH or —(CH₂OH—CH₂OH)_{q}—CH₂CH₃ in which q is an integer ranging from 1 to 10.

According to one preferred embodiment of the invention, the precursor(s) of formula (I) are chosen from tetramethoxyorthosilane (TMOS), tetraethoxyorthosilane (TEOS), dimethyldiethoxysilane (DMDES), (3-mercaptopropyl)trimethoxysilane, (3-aminopropyl)triethoxysilane, N-(3-trimethoxysilylpropyl)pyrrole, 3-(2,4-dinitrophenylamino)propyltriethoxysilane, N-(2-aminoethyl)-3-amino-propyltrimethoxysilane, phenyltriethoxysilane, methyltriethoxysilane and mixtures thereof.

According to one preferred embodiment of the invention, the precursor of formula (I) is chosen from TEOS, a mixture of TEOS and of DMDES in which the DMDES represents from 5 to 30% by weight relative to the TEOS, and TMOS.

The concentration of silica oxide precursor(s) and/or organically modified silica oxide precursors within the aqueous solution is preferably greater than 10% by weight relative to the weight of the aqueous phase. This concentration varies more preferably from 17 to 35% by weight relative to the weight of the aqueous phase.

According to one particular embodiment of the invention, and when the silica or organically modified silica matrix also comprises at least one metal oxide MO₂ in which M is a metal chosen from Zr, Ti, Th, Nb, Ta, V, W and Al, then the aqueous solution of silica oxide or organically modified silica oxide precursor(s) also comprises at least one precursor of said metal oxide, said precursor being chosen from the compounds of the following formula (III):

M(OR⁶)₄ (III)

in which:
M is a metal chosen from Zr, Ti, Th, Nb, Ta, V, W and Al; and
R⁶ is a C₁-C₄ alkyl radical, preferably a methyl or ethyl radical.

The oily phase is preferably formed by one or more compounds chosen from linear or branched alkanes having at least 12 carbon atoms. As an example, dodecane and hexadecane may be mentioned. The oily phase may also be formed by a silicone oil of low viscosity, i.e. lower than 400 centipoise.

The amount of oily phase present within the emulsion may be adjusted according to the desired diameter of the macropores to be obtained for the silica template, it being understood that the higher the oil/water volume fraction, the smaller the diameter of the oil droplets within the emulsion and likewise the smaller the diameter of the macropores.

In general, the oily phase represents from 60 to 90% by volume relative to the total volume of the emulsion. This amount of oil makes it possible to obtain a silica template in which the mean diameter of the macropores varies from 1 to 100 μm approximately.

The surfactant compound may be a cationic surfactant chosen especially from tetradecyltrimethylammonium bromide (TTAB), dodecyltrimethylammonium bromide and cetyltrimethylammonium bromide. When the surfactant compound is cationic, the reaction mixture is brought to a pH of less than 3, preferably less than 1. Tetradecyltrimethylammonium bromide is particularly preferred.

The surfactant compound may also be an anionic surfactant chosen from sodium dodecylsulfate, sodium dodecylsulfonate and sodium dioctylsulfosuccinate (AOT). When the surfactant compound is anionic, the reaction mixture is brought to a pH of greater than 10.

Finally, the surfactant compound may be a nonionic surfactant chosen from surfactants having an ethoxylated head group, and nonylphenols. Among such surfactants, mention may in particular be made of ethylene glycol and propylene glycol block copolymers sold for example under the brand names Pluronic® P123 and Pluronic® F127 by BASF. When the surfactant compound is nonionic, the reaction mixture is brought to a pH of greater than 10 or less than 3, preferably less than 1, and also preferably contains sodium fluoride so as to improve the condensation of the silica oxide precursors.

The total amount of surfactant present within the emulsion may also be adjusted according to the desired diameter of the macropores to be obtained in the silica template. This amount can also vary according to the nature of the surfactant used.

In general, the amount of surfactant varies from 1 to 10% by weight, preferably from 3 to 6% by weight, relative to the total weight of the emulsion.

The step of condensing the silica oxide precursor(s) and/or the organically modified silica oxide precursor(s) is advantageously carried out at a temperature close to room temperature. The duration of this step may vary from a few hours (2 to 3 hours) to a few weeks (2 to 3 weeks) depending on the pH of the reaction medium.

According to one preferred embodiment of the invention, the silica template obtained at the end of the first step is washed with an organic solvent (such as for example tetrahydrofuran, acetone and mixtures thereof) and then dried (for example in air in an oven or by freeze drying) before undergoing the step of being impregnated by the carbon-precursor or ceramic-precursor solution.

The solvent of the coupling agent solution used during the coupling reaction is an organic solvent, preferably chosen from chloroform, toluene and mixtures thereof. Preferably, said solvent is a mixture of equal parts of chloroform and of toluene.

The amount of coupling agent in the solution used for the functionalizing step may be adjusted according to the diameter of the macropores of the silica monolith and the amount of unpurified enzyme that it is desired to immobilize. Generally, this amount may vary from 0.02M to 0.5M, and preferably from 0.05M to 0.2M.

According to one particular and preferred embodiment of the invention, use is made of a 0.1M solution of coupling agent in a 50/50 (w/w) mixture of chloroform and toluene.

The step of functionalizing the cellular monolith with the coupling agent is preferably carried out under vacuum at room temperature for a duration of approximately 72 hours.

The step of immobilizing the unpurified enzyme is preferably carried out under vacuum at room temperature for a duration of approximately 72 hours.

The washing of the monolith at the end of the functionalizing step is preferably carried out with an organic solvent such as, for example, tetrahydrofuran, chloroform or acetone. Finally, the monolith is dried, preferably in air for approximately 2 days.

The washing of the monolith at the end of the step of immobilizing the unpurified enzyme is preferably carried out with distilled water.

The heterogeneous enzymatic catalyst in accordance with the present invention may be used for carrying out chemical reactions that are catalyzed in the heterogeneous phase. The nature of the chemical reactions capable of being catalyzed by the catalyst in accordance with the invention will of course vary depending on the nature of the unpurified enzyme that is immobilized.

Thus, when the unpurified enzyme is a lipase, the catalyst in accordance with the invention is used to catalyze the hydrolysis of fatty acid triglycerides, esterification reactions between an acid and an alcohol, transesterification reactions between an ester and an alcohol, inter-esterification reactions between two esters or transfer reactions of an acetyl group from an ester to an amine or to a thiol.

In particular, when the enzyme is a lipase, said catalyst may be used for example for catalyzing:
the synthesis of butyl oleate, which is a lubricant for biodiesels;
the hydrolysis of glycerol-linoleic ester derivatives to result in soaps or detergents; and
transesterification reactions involved in the synthesis of low-viscosity biodiesels.

The present invention is illustrated by the following embodiment examples, to which the invention is not however limited.

### EXAMPLES

The raw materials used in the following examples are listed below:
98% tetradecyltrimethylammonium bromide (TTAB), from Fluka;
98% tetraethoxyorthosilane (TEOS), from Fluka;
99% dodecane, from Fluka;
Coupling agent: γ-glycidoxypropyltrimethoxysilane sold under the trade name Glymo by Sigma Aldrich (St Louis, Mo.);
Candida rugosa lipase, EC 3.1.1.3, Type VII, at 700 U/mg, from Sigma Chemicals (St Louis, Mo.);
Thermomyces lanuginosus lipase, solution at least 100000 U/g, from Sigma Aldrich (St. Louis, Mo.);
Oleic acid, glyceryl trilinoleate (98%), ethyl linoleate (≧98%), linoleic acid (≧99%), n-heptane, ethanol, 1-butanol, from Sigma Aldrich (Paris, France).
The other chemicals and solvents used in the examples were all of analytical grade or HPLC grade.

These raw materials were used as received from the manufacturers, without additional purification.

Characterizations:

The macroporosity was characterized qualitatively by a scanning electron microscopy (SEM) technique using a scanning electron microscope sold under the reference JSM-840A by the company JEOL, operating at 10 kV. The specimens were coated with gold or carbon in before they were characterized.

The macroporosity was quantified by mercury intrusion measurements using a machine sold under the name Micromeritics Autopore IV, in order to obtain the characteristics of the macroscopic cells making up the monolith backbone.

The specific surface area measurements and the characterizations on the mesoscopic scale were made by nitrogen adsorption-desorption techniques using a machine sold under the name Micromeritics ASAP 2010, the analysis being carried out by BET or BJH calculation methods.

The monoliths were subjected to XRD (X-ray diffraction) analysis with small-angle X-ray scattering (SAXS), using an 18 kW rotating anode X-ray source (Rigaku-200) employing a Ge crystal (111) as monochromator. The scattered radiation was collected on a two-dimensional collector (Imaging Plate system, sold by Mar Research, Hamburg). The distance from the detector to the specimen was 500 mm.

The mesoporosity was characterized qualitatively by a transmission electron microscopy (TEM) technique using a microscope sold under the reference H7650 by the company Hitachi, having an acceleration voltage of 80 kV, and coupled to a camera sold under the reference Orius 11 MPX by the company Gatan Inc.

Analyses by high performance liquid chromatography (HPLC) were carried out on a system equipped with manual injection 600 solvent pumps (Waters, Milford, Mass., USA), in isocratic regime, and using acetonitrile as the mobile phase. The compounds were separated on an Atlantis dC18 (4.6 mm×150 mm, 5 μm) chromatography column with an Atlantis dC18 guard column (Waters). The columns were used at room temperature. Empower® software (Waters) was used for data acquisition and processing. The standards were dissolved in methyl t-butyl ether (MTBE). All the solutions were filtered through a 0.45 μm membrane and degassed before use. The flow rate of the liquid phase was set at 1 ml/min and the volume of the samples injected was 20 μl. The catalyzed esterification reactions were monitored using a refractometer sold under the reference 410 by the company Waters (Milford, Mass., USA). For the detection of the products resulting from catalyzed hydrolysis and transesterification reactions, the system was equipped with an ultraviolet (UV) diode-array detector (WAT996, Waters, Milford, Mass., USA). The measurements were carried out at a wavelength of 204 nm, which corresponded to the maximum absorbance. The following elution gradient was used: (Solvent A: acetonitrile, solvent B: MTBE): A/B: 100/0 (w/w) isocratic for 4 min, A/B:70/30 (w/w) gradient for 2 min, A/B:70/30 (w/w) then A/B: 100/0 (w/w) gradient for 5 min. The column was equilibrated under the conditions given above for 10 minutes.

### Example 1

### Preparation of Silica Monoliths Incorporating an Enzyme

In this example the preparation of silica monoliths and the immobilization of lipases in the macropores of these monoliths are illustrated.

1) First step: Synthesis of the silica monolith (MSi).

5.02 g of TEOS were added to 16.02 g of an aqueous 35 wt % TTAB solution. This solution was then acidified by adding 5.88 g of a 37% concentrated hydrochloric acid solution. The mixture was left to hydrolyze with stirring for around 5 minutes until a single-phase hydrophilic medium (aqueous phase of the emulsion) was obtained. Next, 40.0 g of dodecane (oily phase of the emulsion) were added dropwise to this aqueous phase, with stirring. The mixture was transferred into a cylindrical container acting as a macroscopic mold. The emulsion was then left to condense in the form of a silica monolith for 3 days at room temperature. The silica monolith thus synthesized was then washed three times with tetrahydrofuran (THF). The silica monolith was then dried for 3 days at room temperature and then subjected to a heat treatment at 650° C. for 6 hours, applying a rate of temperature rise of 2° C./min, with a hold at 200° C. for 2 hours. A silica monolith denoted MSi was obtained.

The monolith thus obtained had the following morphological characteristics:
porosity: 94%
density of the monolith: 0.085 g·cm⁻³
density of the silica backbone: 1.56 g·cm⁻³
mesopores of vermiform type, mean distance between 2 walls: 31 Å,
specific surface area: 170 m²·g⁻¹ (BET) signifying a high macroporosity and 45 m²·g⁻¹ (BJH) signifying a low mesoporosity.

2) Second step: Functionalization of the silica monolith with a coupling agent of silane type

The silica monolith obtained above in the preceding step was cut into several pieces of 1 g each.

Each piece was then functionalized with Glymo.

In order to do this, the various pieces of MSi were placed in a container containing a 0.01 mol solution of Glymo in 120 g of chloroform. The suspension was placed under vacuum to promote the impregnation. After 48 hours at room temperature, the suspension was filtered, and then the pieces of MSi recovered were washed with chloroform, then with acetone, before being finally dried in air. Monoliths, the surfaces of the macropores of which were functionalized with Glymo (MSi-Glymo), were thus obtained.

3) Third step: Immobilization of lipases in the MSi-Glymo macropores

540 mg of unpurified Candida rugosa lipase (1CR) were dispersed in 18 ml of distilled water and mixed for one hour until a solution was obtained. Next, 250 mg pieces of MSi-Glymo were introduced into the aqueous lipase solution, and then the mixture was subjected to a 20 mbar vacuum for 72 hours at room temperature in order to impregnate the MSi-Glymo with the lipase solution.

53 mg of unpurified Thermomyces lanuginosus lipase (1TL) were dissolved in 10 ml of distilled water. Next, 350 mg pieces of MSi-Glymo were introduced into the aqueous lipase solution, and then the mixture was subjected to a 20 mbar vacuum for 72 hours at room temperature in order to impregnate the MSi-Glymo with the lipase solution.

The silica monoliths were then extracted from the lipase solutions, then washed three times with distilled water in order to eliminate the excess of enzymes, then dried at room temperature for 12 hours. Silica monoliths that immobilize a lipase by means of Glymo (MSi-Glymo-1CR and MSi-Glymo-1TL) were thus obtained. These monoliths were stored at 4° C. before their use as a heterogeneous enzymatic catalyst.

### Example 2

### Esterification of Oleic Acid by 1-Butanol, in the Presence of an Enzymatic Catalyst in Accordance with the Invention

In this example a catalyzed reaction for the esterification of oleic acid (1) by 1-butanol (2) was carried out according to the following reaction scheme:

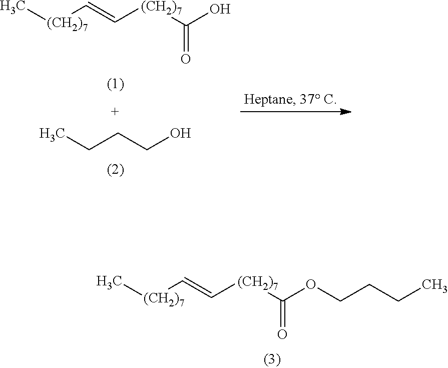

This reaction resulted in the formation of oleic acid butyl ester (3).

2.0 mmol of oleic acid (1), 1.0 mmol of 1-butanol (2) and 0.247 mg of MSi-Glymo-1CR, as prepared above in example 1, were introduced into 2 ml of heptane. The reaction mixture was incubated at 37° C. for 24 hours and the formation of the ester (3) was monitored by HPLC.

The same esterification reaction was thus repeated 21 times using the same catalyst each time. Between the 10^{th} and 11^{th} reactions, the catalyst was stored at 4° C. for 2 months.

Between each esterification reaction, the catalyst was therefore recovered, washed with heptane, and then dried before again being reused to catalyze a new esterification reaction.

The same experiment was carried out, by way of comparison, with a silica monolith as prepared above in example 1 but according to a preparation process in which the step of functionalizing the internal surface of the monolith pores with Glymo was not carried out. The step of impregnating with the Candida rugosa lipase solution was carried out directly after the synthesis of the monolith. An enzymatic catalyst that is not part of the invention and that has been named MSi-1CR was thus obtained.

The results obtained are represented in the appended FIG. 1 in which the degree of conversion of the acid (1) to ester (3), expressed as a percentage, is a function of the time in hours. The number of cycles (one cycle corresponding to carrying out one esterification reaction and one washing of the catalyst) is indicated above each of the peaks. The continuous line plot corresponds to the tracking of the esterification reactions catalyzed by the MSi-Glymo-1CR catalyst in accordance with the invention, whereas the dotted line plot corresponds to the tracking of the esterification reactions carried out with the MSi-LCR catalyst not in accordance with the invention.

The results presented in FIG. 1 demonstrate that the enzymatic catalyst MSi-Glymo-1CR in accordance with the present invention makes it possible to catalyze 19 successive esterification reactions of the acid (1) to ester (3) with a 100% degree of conversion. A slight reduction in the degree of conversion appears between the 20^{th} and 21^{st} cycles. These results also show that the storage of the MSi-Glymo-1CR catalyst at 4° C. for 2 months between the 10^{th} and 11^{th} cycles has not affected its catalytic performances. On the other hand, it is observed that the catalytic activity of the MSi-1CR catalyst not in accordance with the invention never reached 100% (95% only during the first cycle) and that the kinetics of the reaction are furthermore two times slower than with the enzymatic catalyst MSi-Glymo-1CR in accordance with the present invention. Moreover, the catalytic activity of MSi-1CR begins to decrease from the 2″ esterification cycle, reaching 87% at the end of the 5^{th} esterification cycle. These results confirm that even using an unpurified enzyme, the functionalization of the internal surface of the pores of the silica monolith used as a support for the enzyme is necessary for obtaining a good catalytic activity (100% degree of conversion).

In terms of catalytic activity, the MSi-Glymo-1CR catalyst in accordance with the present invention results in degrees of conversion greater than the best degrees of conversion obtained to date with the heterogeneous catalysts described in the prior art, this better catalytic activity being accompanied by a better stability of the catalyst over time. Indeed, the heterogeneous catalysts described in the prior art, in particular the catalysts in which the same enzyme Candida rugosa was immobilized in a polyurethane foam (Awang, R et al., Am. J. Biochem. & Biotech., 2007, 3, 163-166), on natural kaolin (Rahman, M. B. A. et al., Applied Clay Science, 2005, 29, 111-116) or on layered double-metal hydroxides (Raman, M. B. A. et al., Catalysis Today, 2004, 93-95, 405-410) make it possible to achieve a degree of conversion that is only from 70 to 85%, which degree of conversion begins to decrease from the 9^{th} cycle. In this type of catalyst, the mean service life of the immobilized enzyme is furthermore much shorter (around 12 days).

### Example 3

### Hydrolysis of Trilinolein in the Presence of an Enzymatic Catalyst in Accordance with the Invention

In this example the catalyzed hydrolysis reaction of a triester of linoleic acid, trilinolein (4), which is one of the major constituents of olive oil, in water-saturated heptane, was carried out according to the following reaction scheme:

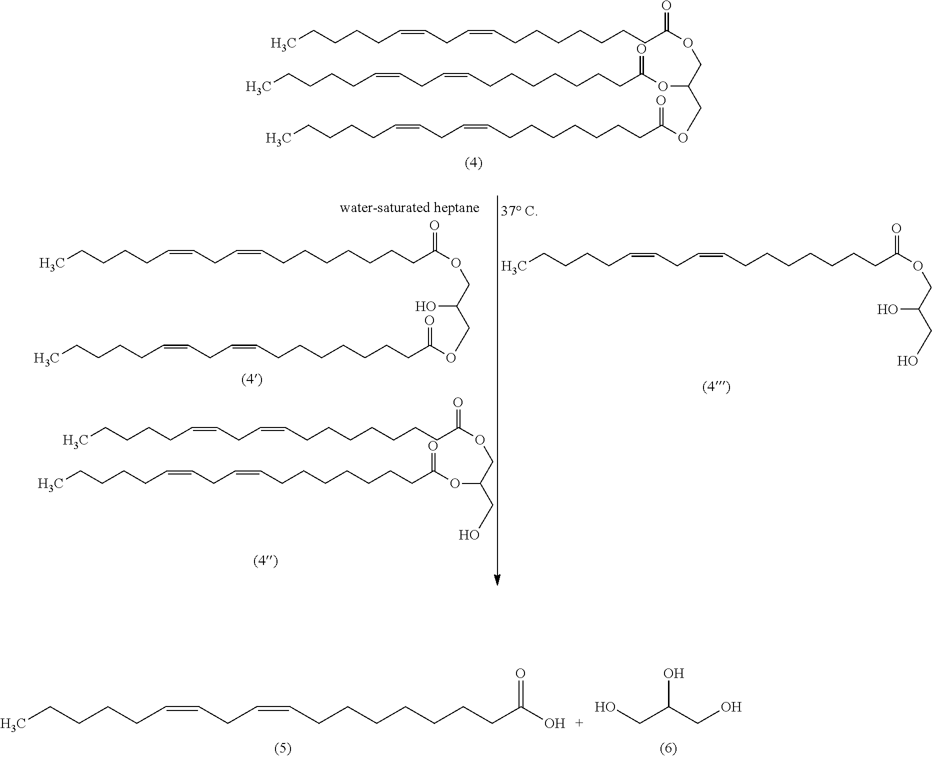

This reaction results in the formation of linoleic acid (5) and glycerol (6); the compounds (4′), (4″) and (4′″) being the intermediate products of the reaction.

0.20 g of MSi-Glymo-1CR as prepared above in example 1, were introduced into 15 ml of water-saturated heptane (0.6% by weight). The mixture was brought to a temperature of 37-38° C., then 100 μl of trilinolein dissolved in MTBE to a concentration of 100 mg/ml were added thereto in order to result, in the end, in a trilinolein solution having a concentration of 0.66 mg/ml. The reaction medium was incubated for 24 hours at a temperature of 37-38° C.

The hydrolysis reaction was monitored by quantifying the disappearance of trilinolein and the appearance of intermediate products (4′), (4″) and (4′″) and end products (linoleic acid (5) and glycerol (6)), by HPLC. After incubating for 24 hours, the heterogeneous MSi-Glymo-1CR catalyst was recovered and then washed 3 times with the water-saturated heptane solution.

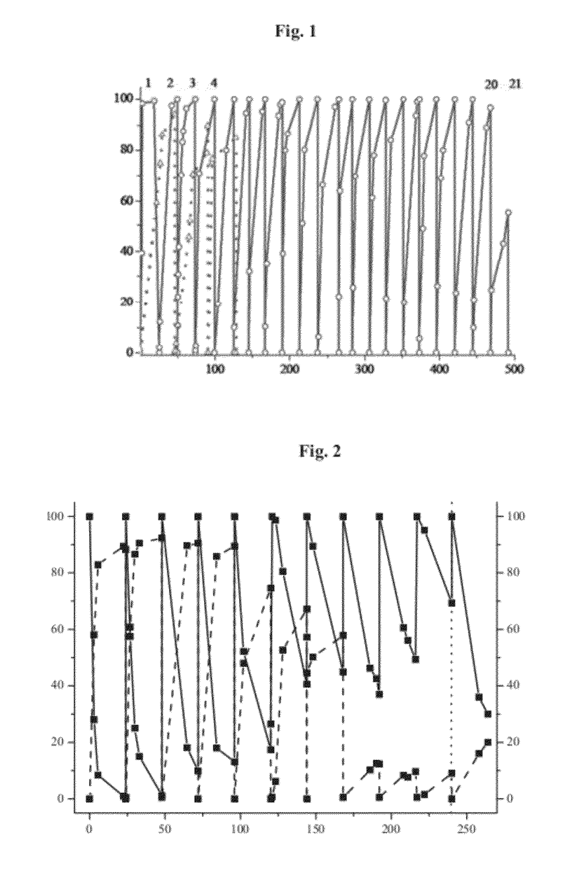

The results obtained are given in the appended FIG. 2 in which the degree of conversion of the triester (4) to linoleic acid (5), expressed as a percentage (vertical left-hand axis, continuous line graph) and the degree of appearance of linoleic acid, expressed as a percentage (vertical right-hand axis, dotted line graph), are a function of the time in hours. The number of cycles (one cycle corresponding to carrying out one hydrolysis reaction and washings of the catalyst) is indicated above the peaks. In this figure, the dotted line located vertically in the 11^{th} cycle indicates that the catalyst was rehydrated in water under vacuum, and then dried in air before being used in a new hydrolysis cycle.

These results show that the heterogeneous MSi-Glymo-1CR catalyst makes it possible to ensure the hydrolysis of trilinolein with a constant degree of conversion of around 65% over 7 cycles at 37° C. This degree of conversion is greater than that which is obtained when the same enzyme is used immobilized on a polypropylene membrane (Deng, H.-T. et al., Enzyme and Microbial. Tech., 2005, 36, 996-1002), and with which a degree of conversion of only 60% is obtained, which drops to 18% after 7 cycles.

These results also demonstrate that such a heterogeneous catalyst can be reused and that the catalytic activity may be increased by rehydration of the catalyst when it begins to decrease, a certain moisture content being necessary for the proper functioning of the lipase.

### Example 4

### Transesterification of Trilinolein in the Presence of an Enzymatic Catalyst in Accordance with the Invention

In this example a catalyzed reaction for the transesterification of trilinolein (4) by ethanol, in a non-aqueous medium, was carried out according to the following reaction scheme:

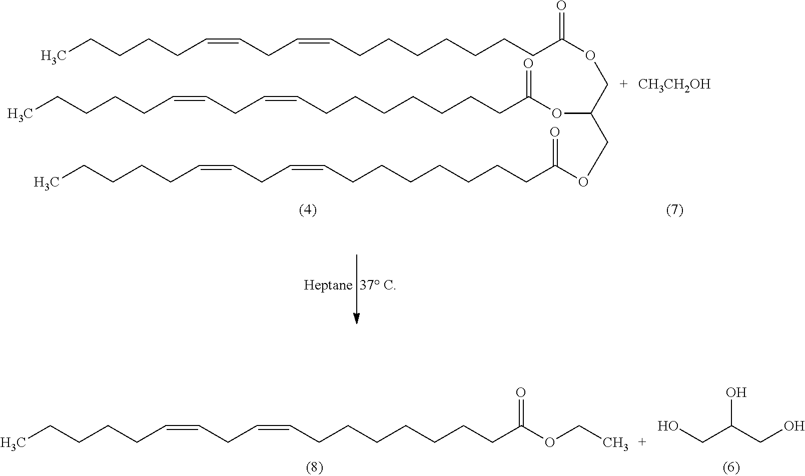

This reaction results in the formation of linoleic acid ethyl ester (8) and glycerol (6). Such a reaction is used for the production of biodiesels, which are methyl or ethyl esters of vegetable oils.

The reaction was carried out in “batch” mode in tubes containing 4 ml of heptane, 100 μl of glyceryl trilinoleate (4) previously dissolved in MTBE to a concentration of 100 mg/ml, 25 mg of ethanol and 387 mg of MSi-Glymo-1TL as prepared above in example 1. The reaction medium was incubated at 37° C. for 24 hours. The conversion of glyceryl trilinoleate (4) to linoleic acid ethyl ester (8) was monitored by HPLC.

Between each transesterification reaction, the catalyst was recovered, washed with heptane, and then dried before again being reused to catalyze a new transesterification reaction.

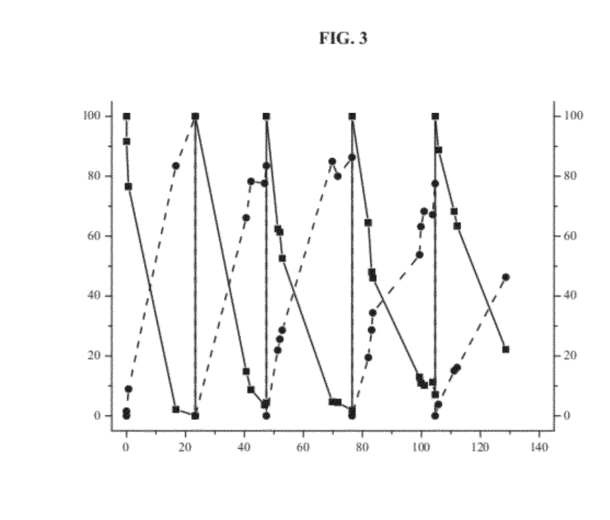

The results obtained are given in the appended FIG. 3 in which the degree of conversion of the triester (4) to ester (8), expressed as a percentage (vertical left-hand axis, continuous line graph) and the degree of production of the ester (8), expressed as a percentage (vertical right-hand axis, dotted line graph), are a function of the time in hours. In this figure, the number of cycles appears above each peak.

These results show that the heterogeneous MSi-Glymo-1TL catalyst may be used for catalyzing the transesterification of trilinolein to linoleic acid ethyl ester with a degree of conversion that reaches 100% in 24 hours at 37° C. during the first cycle and that decreases to 45% at the end of the 5^{th} cycle. However, this loss of activity is certainly due to the presence of the ethanol, which has a denaturing action with respect to the lipase. This result is nevertheless superior to the degree of conversion obtained using a polymer foam modified by macroporous polyglutaraldehyde immobilizing the same enzyme, and which is 90.2% in 24 hours (Dizge, N. et al., Bioresource Technology, 2009, 100, 1983-1991.

Furthermore, these results also demonstrate that the heterogeneous catalyst in accordance with the invention enables the T. lanuginosus lipase to function in an optimal manner in an essentially anhydrous medium (heptane) without it being necessary to add water, whereas it is well known that in order for a lipase to express its maximum catalytic potential, it must be used in a medium having a certain water content (Linko, Y.-Y. et al., JAOCS, 1995, 72(11), 1293-1299).

All of the results presented in these examples demonstrate that the heterogeneous enzymatic catalyst in accordance with the invention make it possible to catalyze various chemical reactions with efficiencies that usually reach 100% of the theoretical catalytic activity, said catalysts being able to be reused a large number of times without significant loss of their catalytic activity.

1. A heterogeneous enzymatic catalyst, wherein said heterogeneous enzymatic catalyst is in the form of a cellular monolith consisting of a silica or organically modified silica matrix, said monolith comprising macropores having a mean size d_{A} of 1 μm to 100 μm, mesopores having a mean size d_{E} of 2 to 50 nm and micropores having a mean size d₁ of 0.7 to 1.5 nm, said pores being interconnected, and in which the internal surface of the macropores is functionalized by a coupling agent chosen from slimes to which an unpurified enzyme is attached, by means of a covalent or electrostatic bond.

2. The catalyst as claimed in claim 1, wherein said monolith has a specific surface area from 200 to 1000 m²/g.

3. The catalyst as claimed in claim 1, wherein the cellular monolith consists of an organically modified silica matrix, the silica bears organic groups R corresponding to the following formula (I):
—(CH₂)ₙ—R¹ (I)
in which:
—0≦n≦5; and
R¹ represents a thiol group, a C₄H₃N— pyrrole group bonded via the nitrogen to the —(CH₂)ₙ— group, an amino group that optionally bears one or more optionally substituted alkyl, alkylamino or an substituents, an alkyl group, a C₂-C₂₁ monohydroxyalkyl or polyhydroxyalkyl group, a phenyl group or a phenyl group substituted by an alkyl radical.

4. The catalyst as claimed in claim 1, wherein the silica matrix of the cellular monolith also comprises one or more metal oxides MO₂ in which M is a metal selected from the group consisting of Zr, Ti, Th, Nb, Ta, V, W and Al.

5. The catalyst as claimed in claim 4, wherein the mixed matrix is a matrix of SiO₂—ZrO₂ type.

6. The catalyst as claimed in claim 1, wherein the coupling agent is chosen from the silanes selected from the group consisting of γ-glycidoxypropyltrimethoxysilane; silyl-containing ionic liquids; the silanes of formula Si(OR²)₃R³ in which R² represents a C₁-C₂ alkyl group, and R³ represents a. —(CH₂OH—CH₂OH)_{q}—CH₂OH or —(CH₂OH—CH₂OH)_{q}—CH₂CH₃ group in which q is an integer ranging from 1 to 10.

7. The catalyst as claimed in claim 6, wherein the coupling agent is γ-glycidoxypropyltrimethoxysilane.

8. The catalyst as claimed in claim 1, wherein the unpurified enzyme is selected from the group consisting of hydrolases, lyases, isomerases and oxidoreductases.

9. The catalyst as claimed in claim 8, wherein the unpurified enzyme is a hydrolase chosen from esterases.

10. The catalyst as claimed in claim 9, wherein the unpurified enzyme is an esterase selected from the group consisting of carboxylic ester hydrolases, aminoacylases, amidases and nitrilases.

11. The catalyst as claimed in claim 10, wherein the unpurified enzyme is selected from the group consisting of Candida rugosa, Candida antarctica, Aspergillus niger, Aspergillus oryzae, Thermomyces lanuginosus, Chromobacterium viscosum, Rhizomucor miehei, Pseudomonas jiuorescens, Pseudomonas cepacia, Penicillium roqueforti, Penicillium expansum and Rhizopus arrhizus lipases and wheat germ lipases.

12. The catalyst as claimed in claim 1, wherein the amount of unpurified enzyme immobilized ranges from 3 to 40% by weight relative to the total weight of the catalyst.

13. A process for preparing a heterogeneous enzymatic catalyst as defined in claim 1, said process comprising;
a first step of preparing a solid silica template in the form of a cellular monolith consisting of a silica or organically modified silica matrix, said monolith comprising macropores having a mean size d_{A} of 1 μm to 100 μm, mesopores having a mean size d_{E} of 2 to 50 nm and micropores having a mean size d_{I} of 0.7 to 1.5 nm, said pores being interconnected, said process further comprises the following steps:
a second step of functionalizing the internal surface of the macropores with a coupling agent chosen from silanes, by vacuum-impregnating the cellular monolith with a solution of the coupling agent in an organic solvent; and
a third step of vacuum-impregnating the thus functionalized monolith with an aqueous solution or an aqueous dispersion of at least one unpurified enzyme.

14. The process as claimed in claim 13, wherein the preparation of the silica template during the first step is carried out according to a process consisting in:
preparing an emulsion by introducing an oily phase into an aqueous surfactant solution;
adding an aqueous solution of at least one silica oxide precursor and/or at least one organically modified silica oxide precursor to the surfactant solution, before or after preparation of the emulsion:
leaving the reaction mixture to rest until said precursor has condensed; and then
drying the mixture in order to obtain the expected solid silica template.

15. The process as claimed in claim 14, wherein the silica oxide or organically modified silica oxide precursor(s) are chosen from silica alkoxides of the following formula (II):
R⁴ₚ(OR⁵)₄₋ₚSi (II)
in which:
R⁴ represents an alkyl radical having 1 to 5 carbon atoms or an aryl radical that optionally bears one or more functional groups;
R⁵ represents an alkyl radical having 1 to 5 carbon atoms or a group of the following formula (I):
—(CH₂)ₙ—R¹ (I)
in which 0≦n≦5, and R¹ is chosen from a thiol group, a C₄H₃N— pyrrole group bonded via the nitrogen to the —(CH₂)ₙ— group, an amino group that optionally bears one or more optionally substituted alkyl, alkylamino or aryl substituents, an alkyl group, a C₂-C₂₁ monohydroxyalkyl or polyhydroxyalkyl group, a phenyl group or a phenyl group substituted by an alkyl radical; and
p is an integer equal to 0, 1, 2 or 3.

16. The process as claimed in claim 15, wherein the precursor(s) of formula (I) are selected from the group consisting of tetramethoxyorthosilane, tetraethoxyorthosilane, dimethyldiethoxysilane, (3-mercaptopropyl)trimethoxysilane, (3-aminopropyl)triethoxysilane, N-(3-trimethoxysilyl-propyl)pyrrole, 3-(2,4-dinitrophenylamino)propyltriethoxysilane, N-(2-aminoethyl)-3-aminopropyltrimethoxysilane, phenyltriethoxysilane, methyltriethoxysilane and mixtures thereof.

17. The process as claimed in claim 16, wherein the precursor of formula (II) is selected from the group consisting of tetraethoxyorthosilane, a mixture of tetraethoxyorthosilane and of dimethyldiethoxysilane in which the dimethyldiethoxysilane represents from 5 to 30% by weight relative to the tetraethoxyorthosilane, and tetramethoxyorthosilane.

18. The process as claimed in claim 13, wherein the silica or organically modified silica matrix also comprises at least one metal oxide MO₂ in which M is a metal chosen from Zr, Ti, Th, Nb, Ta, V, W and Al, and in that the aqueous solution of silica oxide or organically modified silica oxide precursor(s) also comprises at least one precursor of said metal oxide, said precursor being chosen from the compounds of the following formula (III):
M(OR⁶)₄ (III)
in which:
M is a meta chosen from Zr, Ti, Th, Nb, Ta, V, W and Al; and
R⁶ is a C₁-C₄ alkyl radical.

19. The process as claimed in claim 13, the solvent of the coupling agent solution is selected from the group consisting of chloroform, toluene, and mixtures thereof.

20. A method for employing a heterogeneous enzymatic catalyst as defined claim 1, said method comprising the step of:
carrying out chemical reactions that are catalyzed in the heterogeneous phase.

21. The method as claimed in claim 20, wherein the unpurified enzyme is a lipase, for catalyzing the hydrolysis of fatty acid triglycerides, esterification reactions between an acid and an alcohol, transesterification reactions between an ester and an alcohol, inter-esterification reactions between two esters or transfer reactions of an acetyl group from an ester to an amine or to a thiol.

22. The method as claimed in claim 21, for catalyzing:
the synthesis of butyl oleate, which is a lubricant for biodiesels;
the hydrolysis of glycerol-linoleic ester derivatives to result in soaps or detergents; and
transesterification reactions involved in the synthesis of low-viscosity biodiesels.
